(19) United States
(12) Patent Application Publication
(10) Pub. No.: US 2025/0248654 A1
(43) Pub. Date: Aug. 7, 2025

(54) OVERTAPE DESIGNS FOR DEVICES SUCH AS GLUCOSE SENSORS

(71) Applicant: Medtronic MiniMed, Inc., Northridge, CA (US)

(72) Inventors: Boon Khai Ng, Northridge, CA (US); Bo Xiao, Northridge, CA (US); Ravi R. Deverkadra, Northridge, CA (US); Ellis Garai, Woodland Hills, CA (US); Isabella Ella Miya, Northridge, CA (US)

(73) Assignee: Medtronic MiniMed, Inc., Northridge, CA (US)

(21) Appl. No.: 19/040,709

(22) Filed: Jan. 29, 2025

Related U.S. Application Data
(60) Provisional application No. 63/549,373, filed on Feb. 2, 2024.

(51) Int. Cl. A61B 5/00; A61B 5/145; A61M 5/142

(52) U.S. Cl. CPC A61B 5/6833; A61B 5/14532; A61M 5/14248

(57) ABSTRACT

Embodiments of the invention provide skin adhesive patches designed to ease a diabetic patient's maneuverability and alignment of a glucose sensing device as the patient adheres the device to their skin. Embodiments of the invention can be adapted for use with a wide variety of medical components that are coupled to the skin of patients, including insulin infusion sets, patch pumps, and all-in-one patch sets.

### CROSS REFERENCE TO RELATED APPLICATIONS

This application claims the benefit under 35 U.S.C. Section 119(e) of co-pending and commonly-assigned U.S. Provisional Patent Application No. 63/549,373, filed Feb. 2, 2024,entitled “OVERTAPE DESIGNS FOR DEVICES SUCH AS GLUCOSE SENSORS”, which application is incorporated by reference herein.

### TECHNICAL FIELD

Embodiments of the present disclosure generally relate to medical devices, and more particularly, to medical devices or products having a sensor and a transmitter and their associated components including tapes/patches designed to adhere such devices to skin.

### BACKGROUND OF THE INVENTION

Diabetes is a disease in which the body does not produce or properly use insulin. Millions of people in the United States and around the world have been diagnosed with some form of diabetes. Type 1 diabetes results from the body's failure to produce insulin. Type 2 diabetes results from insulin resistance in which the body fails to properly use insulin. In order to effectively manage the disease, diabetics must closely monitor and manage their blood glucose levels through exercise, diet and medications. In particular, both Type 1 and Type 2 diabetics rely on insulin delivery and blood glucose monitoring to control their diabetes.

External infusion devices have been used to deliver medication to a patient as generally described in U.S. Pat. Nos. 6,554,798 and 6,551,276 which are specifically incorporated by reference herein. In addition to delivering medication to a patient, other medical devices have been used to determine body characteristics by obtaining a sample of bodily fluid. A variety of implantable electrochemical sensors have been developed for detecting and/or quantifying specific agents or compositions in a patient's blood. For instance, glucose sensors have been developed for use in obtaining an indication of blood glucose levels in a diabetic patient. Such readings can be especially useful in monitoring and/or adjusting a treatment regimen that typically includes the regular administration of insulin to the patient. Thus, blood glucose readings are particularly useful in improving medical therapies with semi-automated medication infusion pumps of the external type and/or implantable type.

Monitoring blood glucose levels plays an integral role in the management and control of diabetes. Finger stick measurements, glucose sensors and monitors have traditionally been used to check the blood glucose levels of diabetic patients. In recent years, continuous glucose monitoring systems have been developed utilizing the latest sensor technologies incorporating both implantable and external sensors as generally described in U.S. Pat. No. 5,391,250 entitled “Method of Fabricating Thin Film Sensors”, U.S. Pat. No. 6,484,046 entitled “Electrochemical Analyte Sensor,” and U.S. Pat. Nos. 5,390,671, 5,568,806 and 5,586,553, entitled “Transcutaneous Sensor Insertion Set,” all of which are specifically incorporated by reference herein. Newer systems deliver the preciseness of finger stick measurements coupled with the convenience of not having to repeatedly prick the skin to obtain glucose measurements. These newer systems provide the equivalent of over 200 finger stick readings per day. Additionally, continuous glucose monitoring systems allow physicians and patients to monitor blood glucose trends of their body and suggest and deliver insulin based on each patient's particular needs. Accordingly, physicians and medical device companies are always searching for more convenient ways to keep diabetic patients aware of their blood glucose levels throughout the day.

As such, physiological characteristic (or analyte) sensors may be generally used to test analyte levels in patients. For example, thin film sensors may be used for obtaining an indication of blood glucose levels and monitoring blood glucose levels in a diabetic patient. In these instances, a portion of a glucose sensor is positioned subcutaneously/transcutaneously in direct contact with patient extracellular fluid. Glucose sensor readings can be especially useful in adjusting a treatment regimen that typically includes regular administration of insulin to the patient.

A glucose sensor may be packaged and sold as a product that includes certain features or components that allow the patient to adhere, position and subcutaneously/transcutaneously implant the sensor. For example, thin film glucose sensors are often implanted subcutaneously/transcutaneously using an introducer needle, which is packaged with the glucose sensor. The introducer needle is used to puncture the skin of a patient at the same time as the sensor is introduced. The introducer needle is then withdrawn, leaving the sensor in the skin of the patient, with the device typically adhered to the patient by a tape. Once a continuous glucose sensor is inserted, the continuous glucose sensor is designed to monitor glucose concentration of the patient and a sensor signal is produced that is representative of the glucose concentration. The continuous glucose sensor may use wireless data communication techniques to transmit data indicative of the blood glucose levels to a receiving device such as a portable infusion pump, a glucose monitor device, and/or the like. For example, the transmitted sensor signal may be used to generate a controller input for a controller to generate commands that affect the operation of a delivery system to infuse a liquid, which includes insulin, into the patient.

Glucose sensing devices and the like are generally adhered to a patient's skin by a tape and/or adhesive patch. However, conventional adhesive tape/patch designs are difficult for patients to handle and easy for patients to misplace over the device and/or on top of the skin. The misalignment and misplacement of continuous glucose monitoring devices on the skin can result in inadequate coverage and directly impact the therapy duration for the patient. There is a need in the art for adhesive tape and/or patch designs that overcome these problems.

### SUMMARY OF THE INVENTION

As improved continuous glucose monitoring devices allow for longer and longer wear durations, the importance of proper patient placement and adherence of these devices increases significantly. The invention disclosed herein provides skin adhesive patches having features that are designed to increase the ease of patient handling, alignment, application and use of medical devices.

As shown in the Figures, a number of embodiments of the invention disclosed herein are designed to ease the maneuverability and alignment by a diabetic patient during the adherence of a glucose sensing device to the patient's skin. The adhesive patch/overtape features disclosed herein allow patients to easily maneuver and align these adhesive patches/overtapes over devices such as a continuous glucose monitor transmitter. Illustrative features include adhesive patch/overtape and liner elements designed to provide the patient with visual aid and/or tactile/sensory aid and/or gripping and other maneuvering elements to facilitate proper device adhesion to patient skin. In this context, a properly applied adhesive patch/overtape provides additional adhesive strength and protection to secure the device to the skin during use, which is particularly beneficial for glucose sensor survivability/longevity.

The invention disclosed herein has a number of embodiments such as the adhesive patches shown in the figures. Embodiments of the invention include, for example, an adhesive patch comprising: a first flexible layer of material adapted to contact skin; an adhesive composition operatively coupled to the first flexible layer of material; and a central hole disposed in the adhesive patch, wherein the central hole is disposed in a location and the adhesive patch comprises a geometry that allows a user to maneuver the adhesive patch using the central hole with a finger so as to align the adhesive patch with a surface of a medical device, thereby providing the patient with tactile feedback to locate the surface of the medical device as the adhesive patch adheres the medical device to the skin of the patient. Illustrative embodiments of this invention are shown in FIGS. 1-8.

Another embodiment of the invention is an adhesive patch comprising: a first flexible layer of material adapted to contact skin; an adhesive composition operatively coupled to the first flexible layer of material; and a plurality of geometric cut outs disposed in the adhesive patch, wherein the plurality of geometric cut outs are disposed in a location and the adhesive patch comprises a geometry that allows a user to maneuver the adhesive patch using the geometric cut outs with a finger so as to align the adhesive patch with a surface of a medical device and/or the geometric cut outs are disposed in a location that allows visualization of the surface of the medical device, thereby providing the patient with tactile and/or visual feedback to locate the surface of the medical device as the adhesive patch adheres the medical device to the skin of the patient. Illustrative embodiments of this invention are shown in FIG. 2.

Another embodiment of the invention is an adhesive patch comprising: a first flexible layer of material adapted to contact skin; an adhesive composition operatively coupled to the first flexible layer of material; and an adhesion patch liner, wherein the adhesion patch liner is folded and/or comprises a visual pattern and further comprises a central liner cut out patch comprising a cut out cover and disposed on the adhesive patch such that removal of the central liner cut out cover exposes a liner adhesive adapted to allow temporary adhesion to a surface of a medical device so as to align the adhesive patch with a surface of a medical device, thereby providing the patient with feedback to locate the surface of the medical device as the adhesive patch adheres the medical device to the skin of the patient. Optionally the adhesive patch comprises a folded liner disposed on the patch in an orientation selected to allow the patient to remove the liner after the adhesive patch is centered on the medical device. Illustrative embodiments of this invention are shown in FIG. 4.

Another embodiment of the invention is an adhesive patch comprising: a first flexible layer of material adapted to contact skin; an adhesive composition operatively coupled to the first flexible layer of material; and a silhouette printed on the adhesive patch in a location adapted to allow the silhouette to align with a surface of a medical device in a correct orientation, thereby providing the patient with feedback to orient the adhesive patch to the surface of the medical device as the adhesive patch adheres the medical device to the skin of the patient. Optionally the adhesive patch comprises an adhesion patch liner. Illustrative embodiments of this invention are shown in FIGS. 5-6.

Another embodiment of the invention is an adhesive patch comprising: a first flexible layer of material adapted to contact skin; an adhesive composition operatively coupled to the first flexible layer of material; and a preformed architecture feature or shape disposed in the adhesive patch, wherein the preformed architecture feature is configured to mate with a surface or feature of a medical device as the adhesive patch adheres the medical device to the skin of the patient. Illustrative embodiments of this invention are shown in FIG. 7.

In certain embodiments of the invention, the adhesive patch comprises one or more tabs, wherein the one or more tabs are disposed in a location and the adhesive patch comprises a geometry that allowing the patient to maneuver the adhesive patch their with fingers as the adhesive patch adheres the medical device to the skin of the patient. Illustrative embodiments of this invention are shown in FIG. 3. In certain embodiments of the invention, the device comprises one or more tactile (e.g. a textured element) or visual feature that allows the patient to maneuver and align the adhesive patch as the adhesive patch adheres the medical device to the skin of the patient. In certain embodiments of the invention, the adhesive patch is perforated to allow a portion of the patch to be removed by the patient. In some embodiments of the invention, the adhesive patch comprises a plurality of adhesive compositions. In addition, certain embodiments of the invention further comprise a tool adapted to mate with a geometry of the adhesive patch so as to aid patient alignment as the adhesive patch adheres the medical device to the skin of the patient. Illustrative embodiments of this invention are shown in FIG. 8.

In typical embodiments of the invention, the device is a continuous glucose sensor. In some embodiments of the invention, a patient can temporarily mount the adhesive patch to the smartphone, and then use smartphone camera and an Apps as a tool to help the patient align the adhesive patch with the transmitter. In certain embodiments, the adhesive patch and a glucose transmitter are deployed simultaneously, for example by disposing the adhesive patch into an appropriate device element such as a serter.

Embodiments of the invention include methods of making an adhesive patch disclosed herein, comprising forming a patch to exhibit a constellation of elements disclosed herein (e.g., as in the embodiments shown in the figures). For example, one such embodiment comprises A method of making an adhesive patch, the method comprising forming a patch comprising a first flexible layer of material adapted to contact skin; wherein the patch is formed to comprise: an adhesive composition operatively coupled to the first flexible layer of material; and a central hole disposed in the adhesive patch, wherein the central hole is disposed in a location and the adhesive patch comprises a geometry that allows a user to maneuver the adhesive patch using the central hole with a finger so as to align the adhesive patch with a surface of a medical device, thereby providing the patient with tactile feedback to locate the surface of the medical device as the adhesive patch adheres the medical device to the skin of the patient.

Yet another embodiment of the invention includes methods of sensing glucose comprising disposing a glucose sensor on a diabetic patient using the adhesive patch disclosed herein; and sensing glucose using the glucose sensor. Optionally, the adhesive patch is worn by the patient for at least 10 days, at least 15 days, or at least 20 days. In certain embodiments of the invention, the adhesive patch is coupled to an insulin infusion set and/or an infusion pump. Optionally the adhesive patch comprises a therapeutic agent (e.g. a corticosteroid) disposed on the patch.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides diagrams and photos showing illustrative embodiments of skin adhesive patches of the invention and systems in which these patches are used. In this embodiment, the patch comprises a center hole with various geometries allowing patients to maneuver the adhesive patch with a finger and align the adhesive patch with a top surface of a medical device (e.g. a glucose sensor transmitter). These features allow a patient's tactile sensation to locate and align the device surface.

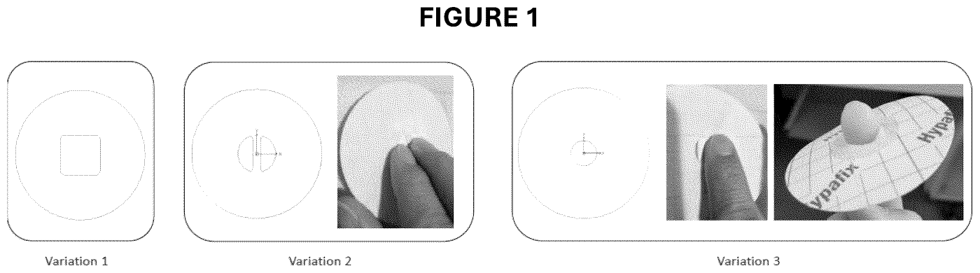

FIG. 2 provides diagrams and photos showing illustrative embodiments of skin adhesive patches of the invention and systems in which these patches are used. In this embodiment, the patch comprises one or more cut-out geometries disposed on the patch to allowing patient to maneuver the skin adhesive patch with a finger and align the skin adhesive patch by visually referring to a top surface of a medical device (e.g. a glucose sensor transmitter). These features use patient's visual sensation to locate a center hole with various geometries allowing patients to maneuver the adhesive patch with a finger and align the adhesive patch with a top surface of a medical device (e.g. a glucose sensor transmitter). These features allow a patient's tactile and/or visual sensation to locate and align the device surface.

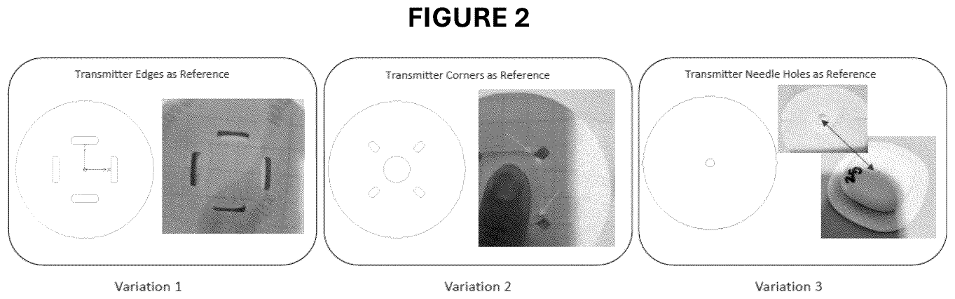

FIG. 3 provides diagrams and photos showing illustrative embodiments of skin adhesive patches of the invention and systems in which these patches are used. In this embodiment, the patch comprises one or more tabs designed to allow patients to maneuver the skin adhesive patch with fingers during skin adhesive patch application.

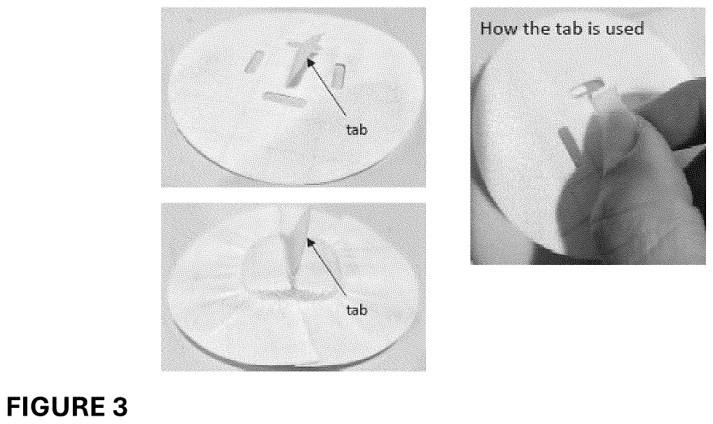

FIG. 4 provides diagrams and photos showing illustrative embodiments of skin adhesive patches of the invention and systems in which these patches are used. In this embodiment, the patch comprises one or more patterned-folded liners on the skin adhesive patch with liner cutout in the middle exposing the skin adhesive patch. The exposed skin adhesive patch has an adhesive which allows temporary adhesion to a top surface of a medical device (e.g. a glucose sensor transmitter). The pattern liners ease the alignment by hanging the skin adhesive patch to the top surface of a medical device (e.g. a glucose sensor transmitter). The folded liners allow patients to easily remove the liner after the skin adhesive patch is centered.

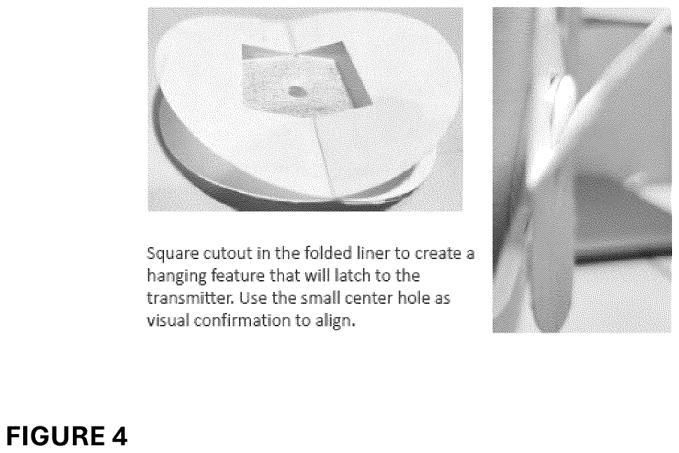

FIG. 5 provides diagrams and photos showing illustrative embodiments of skin adhesive patches of the invention and systems in which these patches are used. In this embodiment, the adhesive patch comprises one or more silhouettes designed to allow patients to help alignment while still allowing for full skin adhesive patch coverage on a top surface of a medical device (e.g. a glucose sensor transmitter).

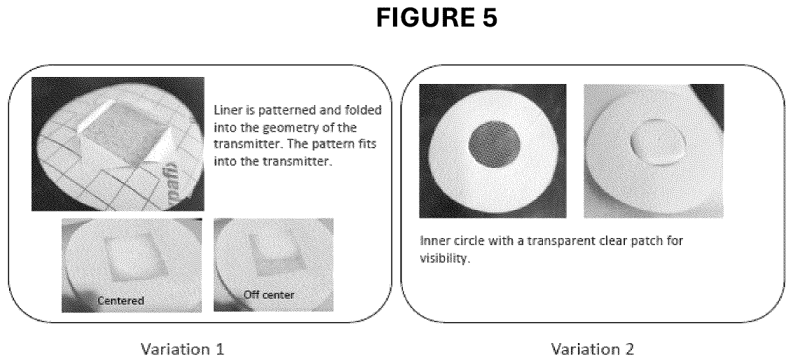

FIG. 6 provides diagrams and photos showing illustrative embodiments of skin adhesive patches of the invention and systems in which these patches are used. In this embodiment, the adhesive patch comprises one or more silhouettes designed to allow patients to help alignment while still allowing for full skin adhesive patch coverage on a top surface of a medical device (e.g. a glucose sensor transmitter).

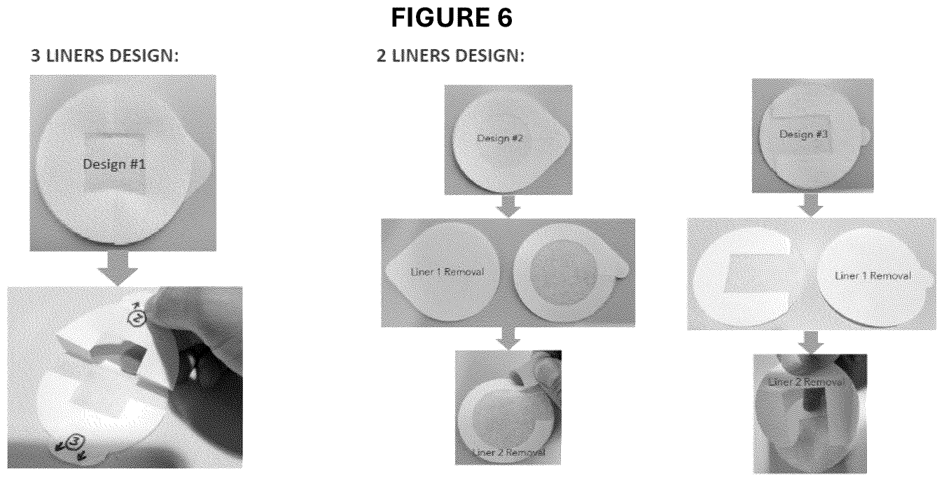

FIG. 7 provides diagrams and photos showing illustrative embodiments of skin adhesive patches of the invention and systems in which these patches are used. In this embodiment, the adhesive patch comprises one or more preformed shapes on the skin adhesive patch to help alignment with a medical device (e.g. a glucose sensor transmitter) while applying the skin adhesive patch.

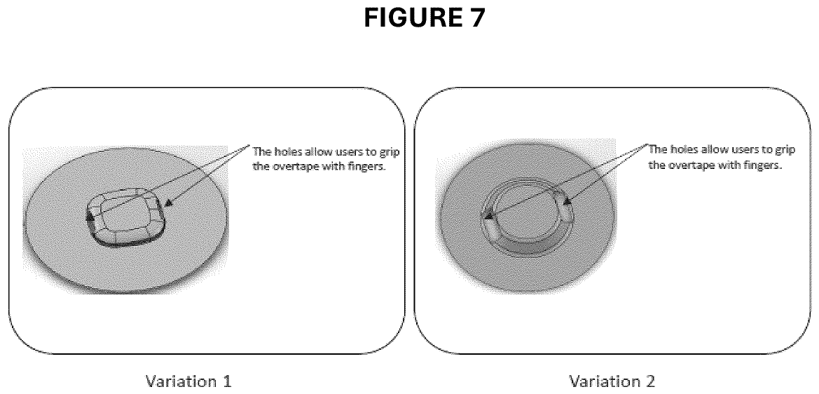

FIG. 8 provides diagrams and photos showing illustrative embodiments of skin adhesive patches of the invention and systems in which these patches are used. In this embodiment, the adhesive patch is combined with a separate tool compatible to the skin adhesive patch design to aid alignment during application.

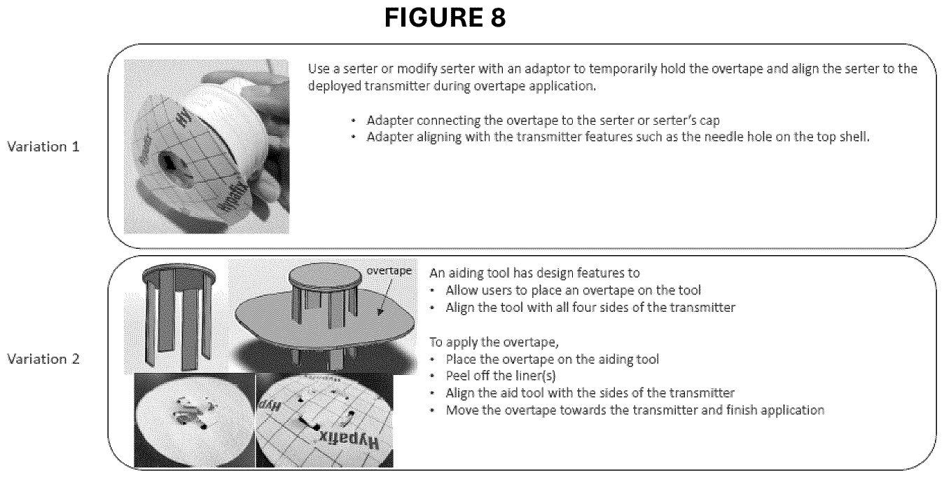

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various publications, patents and published patent specifications are referenced by an identifying citation or by an Arabic numeral, the full citation of which is found preceding the claims. The disclosures of these publications, patents and published patent specifications are hereby incorporated by reference into the present disclosure in their entirety to more fully describe the state of the art to which this invention pertains.

Adhesive patches are commonly used to attach medical components such as glucose sensors and/or infusion sets to the skin of a patient. For example, glucose sensors and/or infusion sets are typically attached to a diabetic wearer's body to monitor glucose levels and/or deliver insulin from an infusion pump to a subcutaneous site on the wearer's body. Medical components such as glucose sensors typically comprise a base and a sensor or cannula inserted into an in vivo site on the wearer's body. In such apparatuses, the base is typically attached to a patch which is attached to the wearer's body with an adhesive system so that glucose sensors and/or other medical components can be removed and replaced periodically.

Many variations on the adhesive patch are practiced in the art, but in general, the adhesive patch comprises a backing layer, a layer of adhesive adjacent the backing layer which faces the wearer, and a release liner over the adhesive to prevent the adhesive from becoming contaminated before use. Adhesives for this purpose are typically provided commercially already applied to a backing layer, with a release liner covering the adhesive.

Embodiments of the invention comprise such adhesive patches that are adapted to couple medical components (e.g., glucose sensors, infusion sets, infusion pumps and the like) to the skin of patients. Embodiments of the invention can be used with a wide variety of medical components/devices such as glucose sensors and/or insulin infusion sets and insulin pumps that are commonly worn by diabetic patients. A continuous glucose monitor comprising a glucose sensor is used as an example of medical components/devices in illustrative embodiments of the invention discussed herein.

The invention disclosed herein has a number of embodiments. Embodiments of the invention include, for example, an adhesive patch comprising an architecture where a first flexible layer of material adapted to contact skin, and an adhesive composition operatively coupled to the first flexible layer of material. In certain embodiments, the adhesive composition is adapted to adhere the first flexible layer of material to skin; and a therapeutic compound is operatively coupled to the adhesive patch such that when the first flexible layer of material is adhered to skin, the therapeutic compound is exposed to the skin such that inflammation is inhibited at a site on skin where the adhesive patch is adhered.

Embodiments of the invention include methods of making an adhesive patch as disclosed herein. The dimensions of the adhesive patch are not critical and may be adapted according to conventional configurations practiced in the art. The patches may be sized as known in the art, with a typical diameter of the patch at the widest point being 1.5 to 2.5 inches, although this dimension likewise is not critical. Apertures, vias and the like through one or more of the layers may be of any shape such as circular, depending on the requirements of receiving a sensor or a cannula, hub or other medical component. The adhesive patches disclosed herein can have additional elements such as tabs adapted to be grasped by the user to manipulate the patch.

Embodiments of the invention further include methods for estimating the concentrations of glucose in vivo, the methods comprising disposing an adhesive patch disclosed herein coupled to a glucose sensor on a diabetic patient; and estimating concentrations of glucose using the glucose sensor. Typically, in these methods, the adhesive patch is worn by the patient for at least 10 days, at least 15 days, or at least 20 days. Optionally in these methods, the patch is coupled to an insulin infusion set and/or an infusion pump.

Embodiments of the invention can use pressure sensitive adhesive compositions. Pressure sensitive adhesives (“PSAs”) based on polymers having acrylic or acrylate moieties are well known in the art and have been used for many years in the medical device arts. Pressure-sensitive acrylic adhesives for application to skin are typically made from compounds such as: 2-ethylhexyl acrylate, isooctyl acrylate or n-butyl acrylate. Polymers in such adhesives can be copolymerized with polar functional monomers such as: acrylic acid, methacrylic acid, vinyl acetate, methyl acrylate, N-vinylcaprolactam, or hydroxyethyl methacrylate (see, e.g., Kenney, J. F., et al. “Medical-grade acrylic adhesives for skin contact.” Journal of applied polymer science 45.2 (1992): 355-361). Other PSAs may be based on silicone. The selection of an appropriate PSA having the desired peel strength may be left to the skill of the person of ordinary skill in this art. Peel strength is a measure of how firmly the PSA adheres to the user's body and may be measured in various ways, measuring the force required to separate two reference surfaces adhered with the PSA. In some embodiments, the PSA is modified by adding one or more active agents, such as a corticosteroid directly to the PSA composition.

It is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims. In the description of the preferred embodiment, reference is made to the accompanying drawings which form a part hereof, and in which is shown by way of illustration a specific embodiment in which the invention may be practiced. It is to be understood that other embodiments may be utilized, and structural changes may be made without departing from the scope of the present invention.

### CONCLUSION

This concludes the description of embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. An adhesive patch comprising:
a first flexible layer of material adapted to contact skin;
an adhesive composition operatively coupled to the first flexible layer of material; and
a central hole disposed in the adhesive patch, wherein the central hole is disposed in a location and the adhesive patch comprises a geometry that allows a user to maneuver the adhesive patch using the central hole with a finger so as to align the adhesive patch with a surface of a medical device, thereby providing the patient with tactile feedback to locate the surface of the medical device as the adhesive patch adheres the medical device to the skin of the patient.

2. An adhesive patch comprising:
a first flexible layer of material adapted to contact skin;
an adhesive composition operatively coupled to the first flexible layer of material; and
a plurality of geometric cut outs disposed in the adhesive patch, wherein the plurality of geometric cut outs are disposed in a location and the adhesive patch comprises a geometry that allows a user to maneuver the adhesive patch using the geometric cut outs with a finger so as to align the adhesive patch with a surface of a medical device and/or the geometric cut outs are disposed in a location that allows visualization of the surface of the medical device, thereby providing the patient with tactile and/or visual feedback to locate the surface of the medical device as the adhesive patch adheres the medical device to the skin of the patient.

3. An adhesive patch comprising:
a first flexible layer of material adapted to contact skin;
an adhesive composition operatively coupled to the first flexible layer of material; and
an adhesion patch liner, wherein the adhesion patch liner is folded and/or comprises a visual pattern and further comprises a central liner cut out patch comprising a cut out cover and disposed on the adhesive patch such that removal of the central liner cut out cover exposes a liner adhesive adapted to allow temporary adhesion to a surface of a medical device so as to align the adhesive patch with a surface of a medical device, thereby providing the patient with feedback to locate the surface of the medical device as the adhesive patch adheres the medical device to the skin of the patient.

4. The adhesive patch of claim 4, wherein the adhesive patch comprises a folded liner disposed on the patch in an orientation selected to allow the patient to remove the liner after the adhesive patch is centered on the medical device.

5. An adhesive patch comprising:
a first flexible layer of material adapted to contact skin;
an adhesive composition operatively coupled to the first flexible layer of material; and
a silhouette printed on the adhesive patch in a location adapted to allow the silhouette to align with a surface of a medical device in a correct orientation, thereby providing the patient with feedback to locate the surface of the medical device as the adhesive patch adheres the medical device to the skin of the patient.

6. The adhesive patch of claim 5, further comprising an adhesion patch liner.

7. An adhesive patch comprising:
a first flexible layer of material adapted to contact skin;
an adhesive composition operatively coupled to the first flexible layer of material; and
a preformed architecture feature disposed in the adhesive patch, wherein the preformed architecture feature is configured to mate with a surface of a medical device as the adhesive patch adheres the medical device to the skin of the patient.

8. The adhesive patch of claim 1, wherein the adhesive patch comprises one or more tabs, wherein the one or more tabs are disposed in a location and the adhesive patch comprises a geometry that allows the patient to maneuver the adhesive patch with their with fingers as the adhesive patch adheres the medical device to the skin of the patient.

9. The adhesive patch of claims 1, wherein the device is a continuous glucose sensor.

10. The adhesive patch of claim 1, further comprising a tool adapted to mate with a geometry of the adhesive patch so as to aid patient alignment as the adhesive patch adheres the medical device to the skin of the patient.

11. A method of making an adhesive patch, the method comprising forming a patch comprising a first flexible layer of material adapted to contact skin; wherein the patch is formed to comprise:
an adhesive composition operatively coupled to the first flexible layer of material; and
a central hole disposed in the adhesive patch, wherein the central hole is disposed in a location and the adhesive patch comprises a geometry that allows a user to maneuver the adhesive patch using the central hole with a finger so as to align the adhesive patch with a surface of a medical device, thereby providing the patient with tactile feedback to locate the surface of the medical device as the adhesive patch adheres the medical device to the skin of the patient.

12. A method of sensing glucose comprising:
disposing a glucose sensor on a diabetic patient using the adhesive patch of claim 1; and
sensing glucose using the glucose sensor.

13. The method of claim 12, wherein the adhesive patch is worn by the patient for at least 10 days, at least 15 days, or at least 20 days.

14. The method of claim 12, wherein the patch is coupled to an insulin infusion set and/or an infusion pump.

15. The method of claim 15, wherein the adhesive patch comprises a corticosteroid selectively patterned on the patch.

16. A method of sensing glucose comprising:
disposing a glucose sensor on a diabetic patient using the adhesive patch of claim 1; and
sensing glucose using the glucose sensor.

17. A method of sensing glucose comprising:
disposing a glucose sensor on a diabetic patient using the adhesive patch of claim 2; and
sensing glucose using the glucose sensor.

18. A method of sensing glucose comprising:
disposing a glucose sensor on a diabetic patient using the adhesive patch of claim 3; and
sensing glucose using the glucose sensor.

19. A method of sensing glucose comprising:
disposing a glucose sensor on a diabetic patient using the adhesive patch of claim 5; and
sensing glucose using the glucose sensor.

20. A method of sensing glucose comprising:
disposing a glucose sensor on a diabetic patient using the adhesive patch of claim 7; and
sensing glucose using the glucose sensor.
